(19) United States
(12) Patent Application Publication
(10) Pub. No.: US 2011/0143386 A1
(43) Pub. Date: Jun. 16, 2011

(54) METHOD FOR THE ANALYSIS OF O-LINKED OLIOSACHARIDES

(75) Inventors: Yehia S. Mechref, Bloomington, IN (US); Milos V. Novotny, Bloomington, IN (US); John A. Goetz, Bloomington, IN (US)

(73) Assignee: Indiana University Research and Technology Corpora, Indianapolis, IN (US)

(21) Appl. No.: 13/058,387

(22) PCT Filed: Aug. 11, 2009
(86) PCT No.: PCT/US09/53385

§ 371 Date: Feb. 10, 2011

Related U.S. Application Data
(60) Provisional application No. 61/090,081, filed on Aug. 19, 2008.

(51) Int. Cl. C12Q 1/37

(52) U.S. Cl. 435/23

(57) ABSTRACT

A method of analyzing O-linked oligosaccharides in a sample is disclosed. The method comprises the steps of digesting a glycoprotein with a proteolytic enzyme, performing solid-phase permethylation of the oligosaccharide, then analyzing the permethylated and non-reduced O-linked oligosaccharides using MALDI-TOF mass spectrometry.

### TECHNICAL FIELD

The present disclosure pertains to the fields of biochemistry and analytical chemistry. More particularly, the present disclosure pertains to a method for analysis of oligosaccharides.

### BACKGROUND

O-glycosylation is a common post-translational modification of proteins. O-linked oligosaccharides play a significant role in development, immunity, infectious diseases and cancer. The functions of O-linked oligosaccharides vary from cell-cell recognition to protein-protein interaction. Many studies of O-linked oligosaccharides have been performed using antibody analysis and nuclear magnetic resonance (NMR). The high sensitivity and scalability of matrix assisted laser desorption ionization time of flight (MALDI-TOF) mass spectroscopy make it an attractive option for the analysis of O-glycans; however, has been limited by sample preparation protocols.

The existing techniques for the analysis of O-glycans rely on microgram amounts of starting material, and they are not feasible in all situations given sample limitations. Therefore, it would be advantageous to develop a technique utilizing sub-microgram levels of glycoproteins which is both simple and reproducible.

Another caveat of the existing chemical cleavage protocols is the associated ‘peeling reactions’ that occur. This further complicates the analysis of simple dimeric and trimeric oligosaccharides, as they could represent a simple structure on the glycoprotein or be the result of a peeling reaction on a larger sugar. The ability to limit or eliminate these reactions will greatly reduce the ambiguity associated with analyzing O-linked oligosaccharides.

Another problem associated with existing chemical cleavage protocols is the tendency for large glycoproteins to mask or bury small O-linked oligosaccharides. We encountered this problem while analyzing human IgA, in which a dimeric oligosaccharide, HexNAc1Hex1, was not observed following a standard chemical cleavage protocol. After digesting the glycoprotein with trypsin, followed by the chemical cleavage protocol, we were able to cleave the dimeric oligosaccharide. Thus, the previously existing techniques for oligosaccharide analysis are found to have disadvantages as described above. We disclose herein a new glycomics technique for analysis of O-linked oligosaccharides with improved sensitivity, reproducibility, and an ability to accommodate smaller sample sizes.

### SUMMARY OF THE INVENTION

A method for analyzing oligosaccharides comprises one or more of the following features or combinations thereof:

According to one illustrative embodiment of the invention, a new glycomics technique is utilized for O-glycan analysis. The technique is a combination of non-specific proteolysis using PRONASE, in combination with solid-phase permethylation, which results in free, permethylated and non-reduced O-linked oligosaccharides.

Glycoproteins are digested with PRONASE at a high ratio of enzyme to protein with a 48 hour reaction time. Samples are then dried and subjected to solid-phase permethylation followed by MALDI analysis. This technique provides a sensitive and reproducible method for glycomic analysis of O-linked oligosaccharides.

In another illustrative embodiment of the invention, the method can be applied for analysis of N-glycans.

### BRIEF DESCRIPTION OF THE DRAWINGS

FIG. 1 shows MALDI-TOF spectra of O-glycans released from 5 μg of intact bovine fetuin (A). The intact fetuin was subjected to permethylation and the released O-glycans were purified by liquid-liquid extraction. The high mass range was examined to determine if N-glycans were released during the procedure and none were detected (B).

FIG. 2 depicts a reaction scheme for the base hydrolysis of O-glycans from a single amino acid.

FIG. 3 shows MALDI-TOF spectra of permethylated O-glycans from either (A) 0.5 μg of fetuin or (B) 2.5 μg of IgA. Samples were first digested with PRONASE for 48 hours and then subjected to spin-column permethylation.

FIG. 4 shows MALDI-TOF spectra of C-GlycoMAP analysis of a PRONASE digestion time course. Fetuin (1 μg) was incubated with PRONASE for either 24 or 48 hours and then subjected to spin-column permethylation.

FIG. 5 shows MALDI-TOF spectra of C-GlycoMAP comparison of O-glycan elimination procedures. 5 μg of Fetuin or 20 μg of IgA were subjected either to 1) 48 hours of PRONASE digestion followed by spin-column permethylation; 2) sodium borohydride method using intact protein; 3) β-elimination using an ammonia-borane complex; or 4) tryptic digestion of the intact protein followed by β-elimination using an ammonia-borane complex. FIG. 5a depicts the C-GlycoMAP spectra of the HexNAcHexNeuNAc structure. FIG. 5b depicts the C-GlycoMAP spectra of the HexNAcHexNeuNAc2 structure. FIG. 5c depicts the C-GlycoMAP spectra of the HexNAc2Hex2NeuNAc2 structure.

FIG. 6 shows MALDI-TOF spectra of O-glycans released from human milk bile-salt-stimulated lipase (BSSL). BSSL (10 μg) was digested using PRONASE for 48 hours and was then subjected to spin-column permethylation.

### DETAILED DESCRIPTION

While the invention is susceptible to various modifications and alternative forms, specific embodiments will herein be described in detail. It should be understood, however, that there is no intent to limit the invention to the particular forms described, but on the contrary, the intention is to cover all modifications, equivalents, and alternatives falling within the spirit and scope of the invention.

In accordance with one embodiment of the invention, a method of analyzing O-linked oligosaccharides is provided. The method comprises the steps of digesting a glycoprotein with a proteolytic enzyme, performing solid-phase permethylation of the oligosaccharide, and analyzing the permethylated and non-reduced O-linked oligosaccharides using MALDI-TOF mass spectrometry.

### Example 1

### Materials and Methods

Chemicals and Materials. Sodium hydroxide, 20-40 mesh beads, 97%, iodomethane (including isotopic versions), 2,5-dihydroxybenzoic acid (DHB) and acetonitrile were acquired from Aldrich (Milwaukee, Wis.). Chloroform and dimethylsulfoxide (DMSO) were obtained from EM Science (Gibbstown, N.J.). Borane-ammonia complex, proteomics-grade trypsin, bovine serum fetuin, human IgA and 28% aqueous ammonium hydroxide were acquired from Sigma Co. (St. Louis, Mo.). PRONASE was obtained from Roche Applied Science (Mannheim, Germany).

The bile-salt-stimulated lipase from human milk was obtained from the Department of Clinical Chemistry, University Hospital, Linkoping, Sweden.

Digestion with PRONASE. The glycoproteins were dissolved in water to a final concentration of 2 mg/ml and PRONASE was added to a final concentration of 0.2 mg/ml. Reaction mixture was then incubated at 55° C. for 48 hours unless otherwise described.

Digestion with Trypsin. The glycoproteins were dissolved in water to a final concentration of 2 mg/ml and trypsin was added to a final ratio of 20:1 protein to enzyme. Reaction mixture was then incubated at 37° C. overnight.

β-Elimination of O-linked Oligosaccharides. Glycoproteins digested using trypsin were subjected to a modified β-elimination protocol. The peptides were first dried using a speed-vac and then had a small volume of 5 mg/ml ammonia borane complex in 28% ammonium hydroxide added to each sample to final concentration of 1 μl per 1 μg of protein. Samples were then incubated for 24 hours at 60° C. After allowing the samples to cool, 1M HCl was added to destroy any residual ammonia borane complex. Finally, the samples were dried and washed three times using methanol prior to solid-phase permethylation.

Permethylation. Solid-phase permethylation was performed using the spin-column technique developed in the lab. Samples were first dissolved in 90 μl of DMSO, 2.7 μl of water and 35 μl of iodomethane. Samples were then passed over a spin-column packed with sodium hydroxide mesh beads a total of eight times. The columns were then washed once with acetonitrile. Chloroform (400 μl) was then added to the samples followed by three 1 ml extractions using 500 mM NaCl. The chloroform layer was saved and dried using a speed-vac and the extracted material was resolubilized using 4 μl of a 50:50 water/methanol mix.

MALDI-TOF Analysis. The permethylated samples were spotted using equal volumes of the sample and a DHB matrix containing 10 mg/ml DHB and 1 mM sodium actetate. Mass spectra were then acquired using an Applied Biosystems 4800 Proteomic Analyzer (Applied Biosystems Inc., Framingham, Mass.). Mass spectra were acquired in positive ion mode.

### Results and Discussion

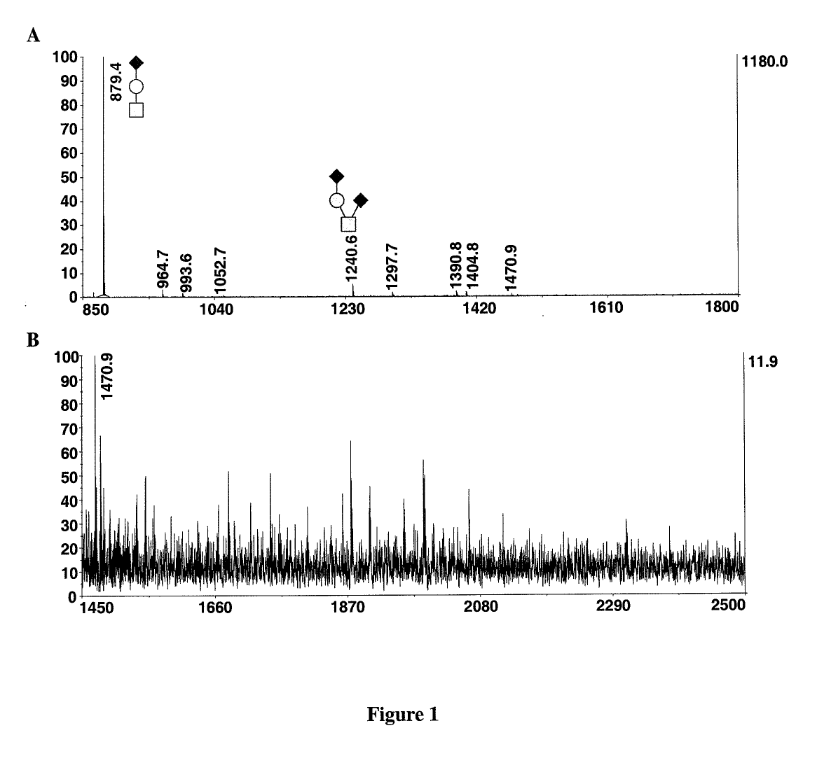

Permethylation of Intact Proteins. We created a new method for the analysis of O-linked oligosaccharides which is an improvement over existing methodologies. High molecular weight proteins have the ability to bury small O-linked oligosaccharides making chemical cleavage procedures inefficient. In the absence of an enzymatic means of cleaving O-linked oligosaccharides these chemical procedures need to be more efficacious to ensure quality data is being collected. The existing chemical cleavage procedures are flawed by another undesirable result, the ability to initiate ‘peeling reactions’. These reactions take place when oligosaccharides undergo cleavages to bonds other than the protein-oligosaccharide link, and the products of these reactions further obfuscate data interpretation. Solid-phase permethylation confers added stability to both N- and O-linked oligosaccharides while improving sensitivity of glycans for MS analysis. The permethylation reaction conditions occur in a highly basic milieu which may have the ability to hydrolyze the linkage between the protein and oligosaccharide. In order to test the ability of the permethylation conditions to release O-linked oligosaccharides, we first began with permethylating intact proteins. FIG. 1A presents the results of the permethylation of intact Fetuin. Two masses which were detected matched those of known Fetuin O-glycans, 879.4 and 1240.6, and the matching masses corresponded to the O-glycans having a non-reduced reducing end. These peaks were then fragmented using both CID and PSD to confirm their composition and it was confirmed that both peaks were O-linked oligosaccharides. A third known O-glycan from Fetuin, having a mass of 1689.8, was not detected. Fetuin is also modified by numerous N-glycan structures, which we were unable to detect in the sample (FIG. 1B), demonstrating that the permethylation procedure is capable of cleaving only O-glycans.

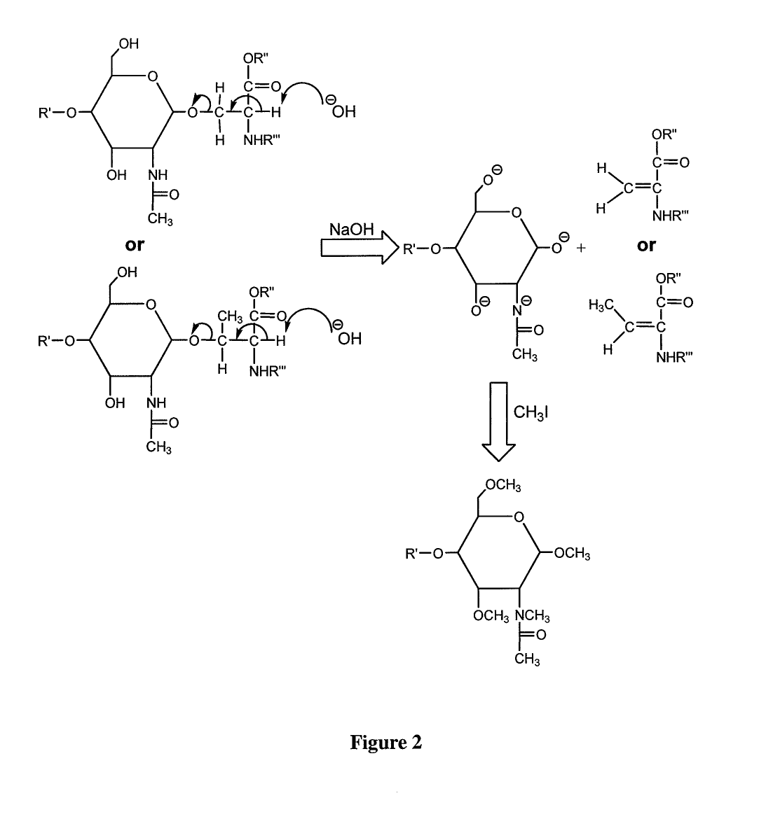

A reaction mechanism has been proposed for the cleavage of O-linked oligosaccharides from the serine or threonine residue (FIG. 2). The basic conditions of the permethylation reaction result in an attack of the hydrogen on the α-carbon of either the serine or threonine. This reaction then results in a rearrangement of the amino acid creating in a double bond between the α-carbon and β-carbon of serine or threonine. This rearrangement results in the cleavage of the bond between the β-carbon and the oxygen molecule on the reducing end of the O-linked oligosaccharide. This cleavage creates an oxygen molecule which is an alkoxide conjugate base in the presence of high levels of sodium hydroxide. This alkoxide conjugate base undergoes permethylation by methyl iodide resulting in the non-reduced reducing end of the O-linked oligosaccharide.

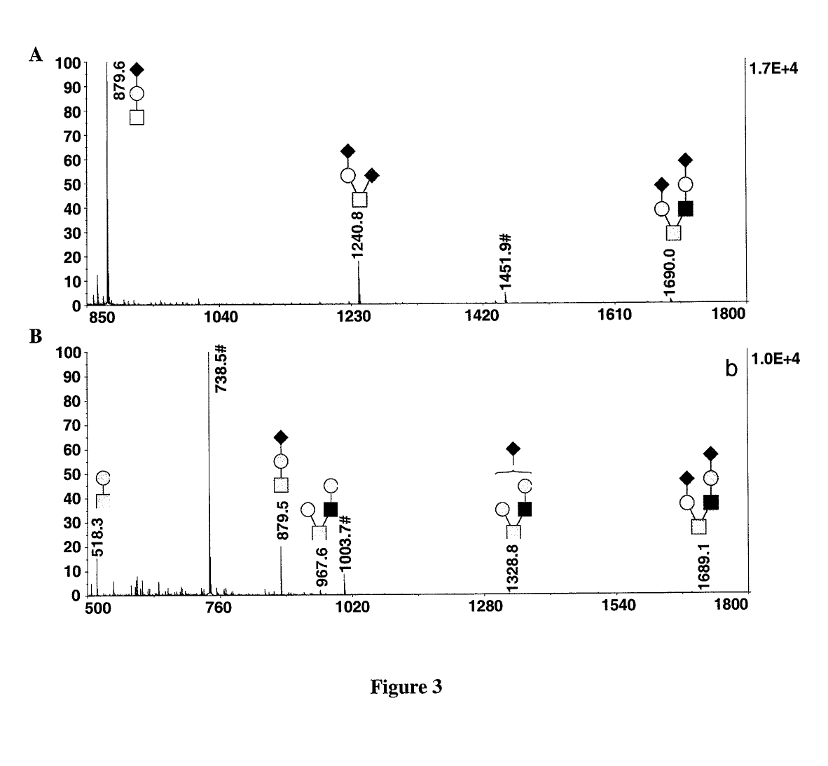

Digestion of Glycoproteins. We improved the ability of the permethylation reaction to cleave O-linked oligosaccharides from glycoproteins. To overcome problems associated with steric interference we used a non-specific protease, PRONASE. Previous work using PRONASE on glycoproteins focused on N-linked oligosaccharides from either standard proteins or bacteria. The study also relied on an alternative method of permethylation which occurs under anhydrous conditions. For the digestion, we utilized ratios of PRONASE to glycoproteins in the 1:1 to 1:10 PRONASE/protein range. For the digestion, we incubated the samples at 55° C. for 24 to 48 hours. After digestion the samples were dried and subjected to solid-phase permethylation using the spin column technique. As seen in FIG. 3A, we have permethylated 500 ng of calf serum fetuin and performed MS analysis on 10% of the sample, approximately 50 ng. We can see peaks with masses corresponding to all three O-glycans present on calf serum fetuin, 879.4, 1240.6, 1689.8. The three O-glycans correspond in both size and relative intensity to previously published results using calf serum fetuin. This demonstrates that the digestion facilitates the cleavage of the O-glycans from the amino acid via the base hydrolysis mechanism. When using intact proteins, we were unable to cleave the largest O-glycan with a mass of 1689.8 from calf serum fetuin. This O-linked oligosaccharide is present using the digestion-permethylation method. Another caveat of previously described methods of O-linked oligosaccharide cleavage is the ability of large proteins which may have relatively few sites for glycosylation to bury or mask linkage sites making them insensitive to chemical cleavages. Therefore, we wanted to use this technique on a larger protein with potentially fewer sites of glycosylation. In FIG. 3B, we have performed the same analysis using human IgA, using an equal molar value of the protein compared to calf serum fetuin. The results show O-glycans with masses of 518.3, 879.5, 967.6, 1328.8 and 1689.1 which also correspond to previous experiments using human IgA. This new technique allowed for the detection of more O-glycan than other groups had previously reported. To demonstrate the consistency of the technique we performed this identical analysis six times and compiled the data. The data was processed using the PeakCalc software and relative intensities were generated. The relative intensity data was subjected to statistical analysis to determine the average value of the relative intensity, the standard deviation, the standard error of the mean and the relative standard deviation. The results are shown in Table 1, demonstrating that the standard deviation of the samples is quite low, especially for O-glycans with a higher relative intensity.

TABLE 1

| Mass | Average | STDEV | SEM | RSD[%] |
|---|---|---|---|---|
| 879.4 | 77.28 | 1.43 | 0.58 | 1.85 |
| 1240.6 | 20.83 | 1.37 | 0.56 | 6.59 |
| 1689.8 | 1.89 | 0.30 | 0.12 | 15.67 |

Human IgA 5 μg n = 6

| | | | | |
|---|---|---|---|---|
| 518.4 | 36.98 | 2.88 | 1.18 | 7.79 |
| 879.4 | 56.76 | 3.37 | 1.37 | 5.93 |
| 967.6 | 3.91 | 0.41 | 0.17 | 10.46 |
| 1328.8 | 2.02 | 0.27 | 0.11 | 13.49 |
| 1689.8 | 0.32 | 0.09 | 0.04 | 28.67 |

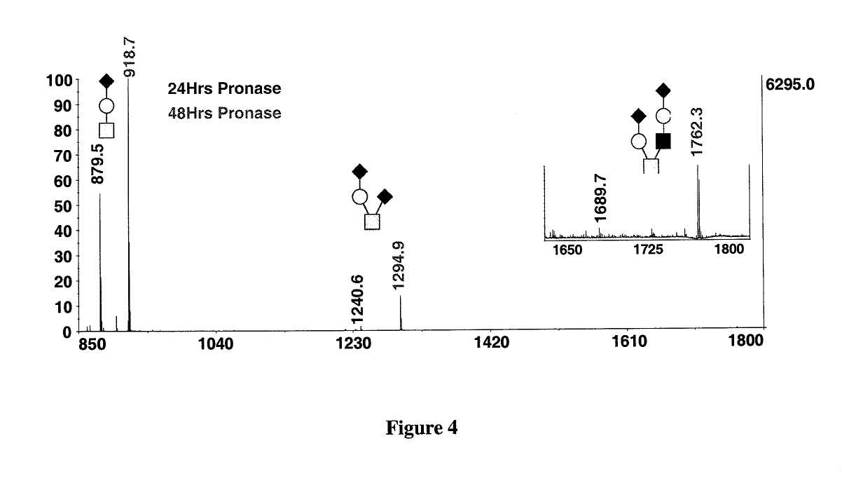

The O-glycans with the lowest relative intensities did have a much higher RSD then glycans with greater relative intensities, a statistical issue that is difficult to resolve. We have demonstrated that this technique is both robust and reliable when compared to previously published methods. To investigate the role of the PRONASE digestion, we performed a time course experiment using calf serum fetuin. Samples were incubated for either 24 or 48 hours in the presence of PRONASE and then dried to completion. After solid phase permethylation using the C-GlycoMAP method, we were able to directly compare the results from the two time points. The mass sepectrum, seen in FIG. 4, indicates that the larger O-linked oligosaccharides are more sensitive to the PRONASE digestion. More than 50% of the relative intensity was maintained for the first O-glycan when comparing the 24 to 48 hour digestion, peaks 879.5 and 918.7. However with the larger O-glycans, less then 20% was able to be cleaved after 24 hours when compared to 48 hours. Presumably, this results from the smaller O-linked oligosaccharides being more susceptible to the base hydrolysis cleavage. This result was also confirmed by the intact protein permethylation in which we only saw the smaller O-glycans.

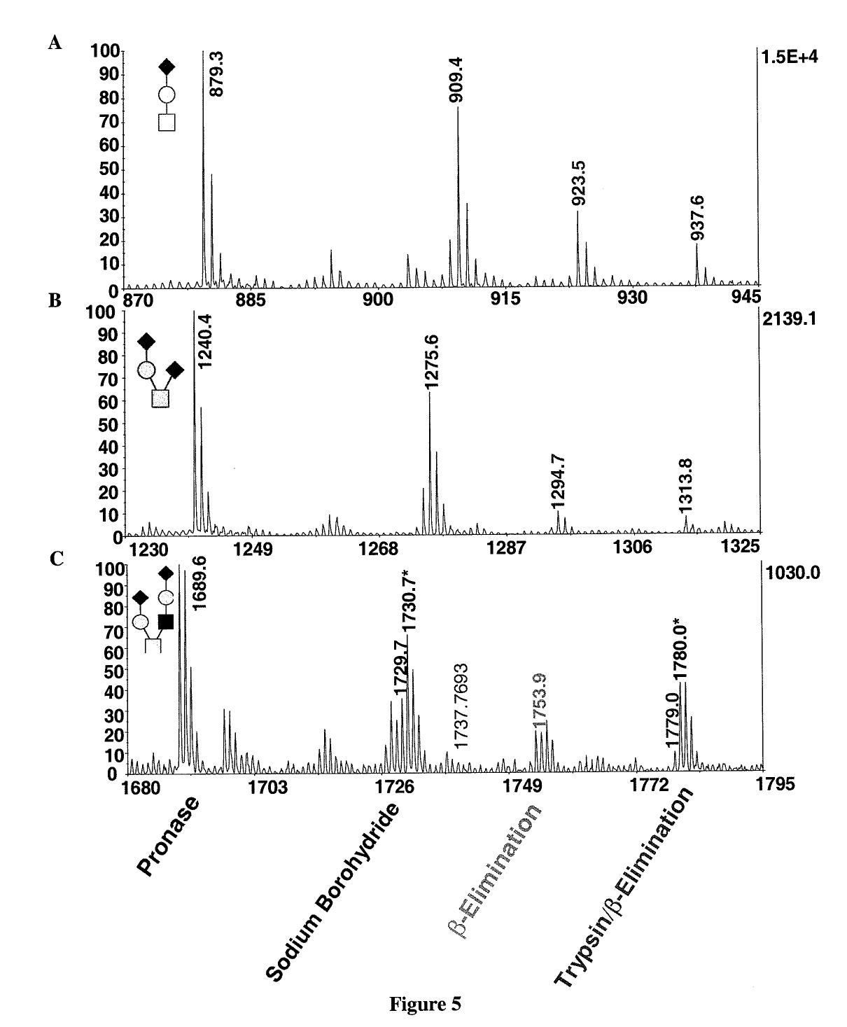

C-GlycoMAP Comparison of O-Glycan Cleavage Techniques. We performed a direct comparison of the β-elimination technique and the PRONASE/permethylation technique of O-glycan cleavage using C-GlycoMAP. To facilitate this comparison we utilized equal masses of both calf serum fetuin and human IgA and subjected them to three different protocols. The first sets of proteins subjected to the PRONASE/permethylation protocol were permethylated using the CH₃I methyl iodide. The second sets of proteins were subjected to the previously described method of β-elimination using ammonia-borane complex and the resulting sugars were then permethylated using CHD₂I methyl iodide. The final sets of proteins were subjected to a modified method of the β-elimination technique in which the proteins were first digested using trypsin and then subjected to the ammonia-borane complex. These final sets of O-linked oligosaccharides were then permethylated using CD₃I methyl iodide. These samples were then pooled and analyzed together using MALDI. As seen in FIG. 5A, the resulting O-glycans from calf serum fetuin indicate that the PRONASE/permethylation technique yields significantly higher peaks, which are still consistent with the known levels of these O-glycans, when compared to either standard β-elimination or the modified β-elimination protocol. Interestingly, we see that the permethylation procedure is still cleaving the O-linked oligosaccharides from the calf serum fetuin in both the standard β-elimination and the modified β-elimination protocol resulting in the peaks marked with an asterisk. This can be attributed to the apparent lability of this modification on fetuin as permethylation alone was able to cleave O-glycans from the intact protein. The data for human IgA, as seen in FIG. 5B, is more compelling when making an argument against the use of previous published procedures. The standard β-elimination technique yields virtually no O-glycans, and the modified technique improves only slightly at cleaving the O-linked oligosaccharides. When comparing peak intensity of identical O-glycans using the different techniques, we see a drop of between 75-90% from the PRONASE/permethylation technique to either of the β-elimination protocols.

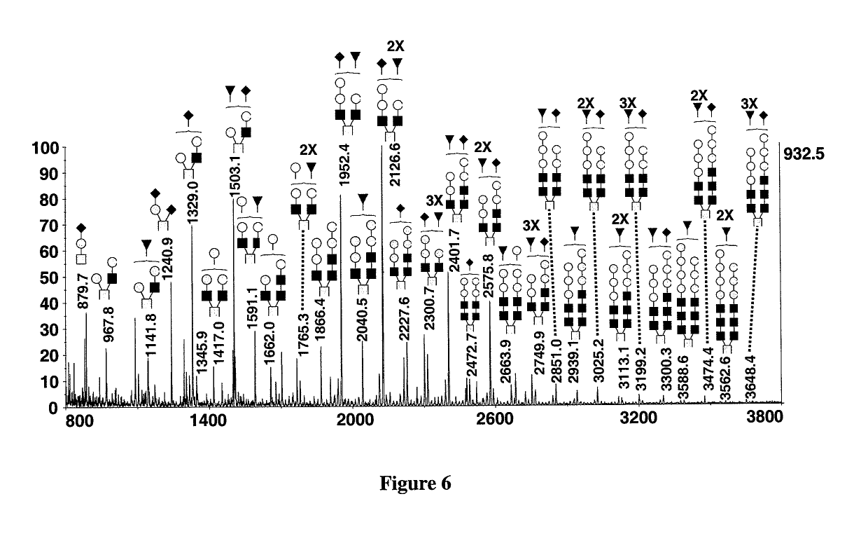

Profiling O-glycans on a Complex Sample. In order to demonstrate the potential of this technique we analyzed the O-linked oligosaccharides on a complex protein, bile-salt-stimulated lipase (BSSL) from human milk. This enzyme is a 100 kDa protein that is known for having a region in its C-terminus which contains many serine and threonine residues resulting in a complex web of O-glycosylation. As seen in FIG. 6, we were able to use 1 μg of BSSL and identify more than thirty separate O-glycans using the PRONASE/permethylation technique. Consistent with previous reports, we see a high level of both fucosylation and sialylation of the O-glycans of BSSL. This demonstrated that the PRONASE-permethylation technique was capable of cleaving large and complex O-glycans without damaging them through peeling reactions. Furthermore, these samples can now be separated in order to gain more information about the structure and linkages of the O-glycans on BSSL or any other protein with a complex level of O-glycosylation.

TABLE 2

| Obs. | Calc. | | |
|---|---|---|---|
| Mass | Mass | ΔMass | Composition |
| 866.704 | 866.437 | 0.268 | Hex₁HexNAc₁Deoxyhexose₂ |
| 879.677 | 879.432 | 0.245 | Hex₁HexNac₁NeuAc₁ |
| 896.711 | 896.447 | 0.264 | Hex₂HexNAc₁Deoxyhexose₁ |
| 937.745 | 937.474 | 0.271 | Hex₁HexNAc₂Deoxyhexose₁ |
| 967.763 | 967.484 | 0.279 | Hex₂HexNAc₂ |
| 1083.816 | 1083.532 | 0.284 | Hex₂HexNAc₁NeuAc₁ |
| 1124.857 | 1124.558 | 0.299 | Hex₁HexNAc₂NeuAc₁ |
| 1130.871 | 1130.558 | 0.313 | Hex₄HexNAc₁ |
| 1141.858 | 1141.573 | 0.285 | Hex₂HexNAc₂Deoxyhexose₁ |
| 1171.880 | 1171.584 | 0.297 | Hex₃HexNAc₂ |
| 1212.930 | 1212.611 | 0.32 | Hex₂HexNAc₃ |
| 1240.901 | 1240.606 | 0.295 | Hex₁HexNAc₁NeuAc₂ |
| 1298.979 | 1298.647 | 0.333 | Hex₁HexNAc₂Deoxyhexose₁NeuAc₁ |
| 1315.983 | 1315.663 | 0.32 | Hex₂HexNAc₂Deoxyhexose₂ |
| 1328.989 | 1328.658 | 0.331 | Hex₂HexNAc₂NeuAc₁ |
| 1345.986 | 1345.673 | 0.313 | Hex₃HexNAc₂Deoxyhexose₁ |
| 1387.031 | 1386.700 | 0.331 | Hex₂HexNAc₃Deoxyhexose₁ |
| 1417.059 | 1416.710 | 0.349 | Hex₃HexNAc₃ |
| 1490.072 | 1489.752 | 0.32 | Hex₂HexNAc₂Deoxyhexose₃ |
| 1503.108 | 1502.747 | 0.361 | Hex₂HexNAc₂Deoxyhexose₁NeuAc₁ |
| 1520.190 | 1519.762 | 0.428 | Hex₃HexNAc₂Deoxyhexose₂ |
| 1591.173 | 1590.800 | 0.373 | Hex₃HexNAc₃Deoxyhexose₁ |
| 1621.210 | 1620.810 | 0.401 | Hex₄HexNAc₃ |
| 1662.218 | 1661.837 | 0.381 | Hex₃HexNAc₄ |
| 1677.214 | 1676.836 | 0.378 | Hex₂HexNAc₂Deoxyhexose₂NeuAc₁ |
| 1690.224 | 1689.832 | 0.392 | Hex₂HexNAc₂NeuAc₂ |
| 1748.277 | 1747.873 | 0.404 | Hex₂HexNAc₃Deoxyhexose₁NeuAc₁ |
| 1765.294 | 1764.889 | 0.405 | Hex₃HexNAc₃Deoxyhexose₂ |
| 1778.276 | 1777.884 | 0.392 | Hex₃HexNAc₃NeuAc₁ |
| 1795.324 | 1794.899 | 0.425 | Hex₄HexNAc₃Deoxyhexose₁ |
| 1819.284 | 1818.911 | 0.373 | Hex₂HexNAc₄NeuAc₁ |
| 1836.337 | 1835.926 | 0.411 | Hex₃HexNAc₄Deoxyhexose₁ |
| 1866.365 | 1865.936 | 0.429 | Hex₄HexNAc₄ |
| 1922.422 | 1921.963 | 0.46 | Hex₂HexNAc₃Deoxyhexose₂NeuAc₁ |
| 1939.428 | 1938.978 | 0.45 | Hex₃HexNAc₃Deoxyhexose₃ |
| 1952.414 | 1951.973 | 0.441 | Hex₃HexNAc₃Deoxyhexose₁NeuAc₁ |
| 1982.412 | 1981.984 | 0.428 | Hex₄HexNAc₃NeuAc₁ |
| 2010.451 | 2010.015 | 0.436 | Hex₃HexNAc₄Deoxyhexose₂ |
| 2040.473 | 2040.026 | 0.447 | Hex₄HexNAc₄Deoxyhexose₁ |
| 2070.487 | 2070.036 | 0.451 | Hex₅HexNAc₄ |
| 2098.537 | 2098.031 | 0.507 | Hex₄HexNAc₂NeuAc₂ |
| 2113.509 | 2113.067 | 0.442 | Hex₃HexNAc₃Deoxyhexose₄ |
| 2126.550 | 2126.062 | 0.488 | Hex₃HexNAc₃Deoxyhexose₂NeuAc₁ |
| 2156.534 | 2156.073 | 0.462 | Hex₄HexNAc₃Deoxyhexose₁NeuAc₁ |
| 2214.620 | 2214.115 | 0.505 | Hex₄HexNAc₄Deoxyhexose₂ |
| 2227.612 | 2227.110 | 0.502 | Hex₄HexNAc₄NeuAc₁ |
| 2268.605 | 2268.137 | 0.469 | Hex₃HexNAc₅NeuAc₁ |
| 2300.666 | 2300.152 | 0.514 | Hex₃HexNAc₃Deoxyhexose₃NeuAc₁ |
| 2315.688 | 2315.163 | 0.526 | Hex₅HexNAc₅ |
| 2388.711 | 2388.204 | 0.507 | Hex4HexNAc4Deoxyhexose3 |
| 2401.743 | 2401.199 | 0.545 | Hex4HexNAc4Deoxyhexose1NeuAc1 |
| 2472.763 | 2472.236 | 0.527 | Hex4HexNAc5NeuAc1 |
| 2489.780 | 2489.252 | 0.528 | Hex5HexNAc5Deoxyhexose1 |
| 2575.848 | 2575.289 | 0.56 | Hex4HexNAc4Deoxyhexose2NeuAc1 |
| 2588.894 | 2588.284 | 0.61 | Hex4HexNAc4NeuAc2 |
| 2663.916 | 2663.341 | 0.575 | Hex5HexNAc5Deoxyhexose2 |
| 2676.881 | 2676.336 | 0.546 | Hex5HexNAc5NeuAc1 |
| 2749.968 | 2749.378 | 0.59 | Hex4HexNAc4Deoxyhexose3NeuAc1 |
| 2762.974 | 2762.373 | 0.601 | Hex4HexNAc4Deoxyhexose1NeuAc2 |
| 2837.980 | 2837.430 | 0.55 | Hex5HexNAc5Deoxyhexose3 |
| 2851.030 | 2850.425 | 0.605 | Hex5HexNAc5Deoxyhexose1NeuAc1 |
| 2939.115 | 2938.478 | 0.638 | Hex6HexNAc6Deoxyhexose1 |
| 3025.188 | 3024.515 | 0.674 | Hex5HexNAc5Deoxyhexose2NeuAc1 |
| 3113.156 | 3112.567 | 0.589 | Hex6HexNAc6Deoxyhexose2 |
| 3126.225 | 3125.562 | 0.664 | Hex6HexNAc6NeuAc1 |
| 3199.228 | 3198.604 | 0.624 | Hex5HexNAc5Deoxyhexose3NeuAc1 |
| 3300.345 | 3299.652 | 0.693 | Hex6HexNAc6Deoxyhexose1NeuAc1 |
| 3388.273 | 3387.704 | 0.569 | Hex7HexNAc7Deoxyhexose1 |
| 3474.413 | 3473.741 | 0.673 | Hex6HexNAc6Deoxyhexose2NeuAc1 |
| 3648.464 | 3647.830 | 0.634 | Hex6HexNAc6Deoxyhexose3NeuAc1 |

### CONCLUSIONS

The current methods that exist for the analysis of O-linked oligosaccharides have left much room for improvement. In the absence of a consistent enzymatic release of O-linked oligosaccharides there is a need to develop techniques which mimic these results. We have developed a method of O-linked oligosaccharide cleavage that utilizes non-specific proteolysis in combination with solid-phase permethylation which results in free non-reduced O-glycans. This allows further processing of the samples to include separation using liquid chromatography or direct infusion for analysis using mass spectrometry. The result is an inexpensive, consistent and more sensitive technique which allows minimal processing of the samples. Furthermore, the technique does not increase the amount of time a sample is processed. This technique also eliminates the use of several potentially hazardous chemicals when compared to existing β-elimination techniques. This method provides the ability to perform analysis of O-linked oligosaccharides on samples previously thought to be impossible given sample size limitations or buffer related complications.

While the invention has been illustrated and described in detail in the foregoing description, such an illustration and description is to be considered as exemplary and not restrictive in character, it being understood that only the illustrative embodiments have been described and that all changes and modifications that come within the spirit of the invention are desired to be protected. Those of ordinary skill in the art may readily devise their own implementations that incorporate one or more of the features described herein, and thus fall within the spirit and scope of the present invention.

We claim:

1. A method for analyzing oligosaccharides in a sample comprising the steps of:
digesting a glycoprotein enzymatically to cleave an oligosaccharide;
permethylating the oligosaccharide; and
conducting MALDI-TOF mass spectrometry on the oligosaccharide.

2. The method of claim 1 wherein the oligosaccharide is an O-linked oligosaccharide.

3. The method of claim 2 wherein the oligosaccharide is an N-linked oligosaccharide.

4. The method of claim 1 wherein said digesting step is carried out using a non-specific protease or proteases.

5. The method of claim 1 wherein said permethylating step is solid-phase permethylation.

6. The method of claim 4 wherein the ratio of protease or proteases to protein is 1:1 to 1:10.

7. The method of claim 5 wherein said permethylation occurs under anhydrous conditions.
